(19) United States
(12) Patent Application Publication
(10) Pub. No.: US 2023/0157297 A1
(43) Pub. Date: May 25, 2023

(54) ANTIVIRAL AND ANTIBACTERIAL COMPOSITION

(71) Applicant: Cedar Advanced Technology Group Ltd., Zug (CH)

(72) Inventors: Said FARHA, Thalwil (CH); Kamal FARHA, Pleasantville, NY (US); Gerald A. HUTCHINSON, Austin, TX (US); Bradley J. NELSON, Zumikon (CH); Salvador PANÉ VIDAL, Zurich (CH); Alexei ERMAKOV, Piscataway, NJ (US); Carlos FRANCO PUJANTE, Zurich (CH); Nuria IZQUIERDO USEROS, Barcelona (ES); Julian BLANCO ARBUES, Vic (ES)

(73) Assignee: Cedar Advanced Technology Group Ltd., Zug (CN)

(21) Appl. No.: 17/920,564

(22) PCT Filed: Apr. 22, 2021
(86) PCT No.: PCT/EP2021/060580

§ 371 Date: Oct. 21, 2022

Related U.S. Application Data
(60) Provisional application No. 63/014,540, filed on Apr. 23, 2020.
(60) Provisional application No. 63/036,317, filed on Jun. 8, 2020.
(60) Provisional application No. 63/058,407, filed on Jul. 29, 2020.

(51) Int. Cl. A01N 47/44; A01N 37/36; A01N 37/44; A01N 25/10; A01P 1/00; A01N 43/50

(52) U.S. Cl. CPC A01N 47/44; A01N 37/36; A01N 37/44; A01N 25/10; A01P 1/00; A01N 43/50

(57) ABSTRACT

The use of a composition as antiviral and/or antimicrobial agent, wherein the composition includes at least a positively charged natural or unnatural amino acid, an organic acid, a cationic polymer and a zwitterionic surfactant.

The present invention relates to the use of a composition as antiviral and/or antimicrobial agent.

Coronaviruses (CoVs) are enveloped positive RNA viruses, belonging to the coronaviridae family and the order

Nidovirales. They are capable of adapting to new environments through mutation and recombination and are programmed to alter host range and tissue tropism. Coronaviruses are phylogenetically subdivided into four genera, α, β, γ, and δ, with type α and β known to be able to infect humans. Coronavirus β can be classified as Severe Acute Respiratory Syndrome (SARS) and Middle East Respiratory Syndrome (MERS), both considered as zoonotic infections. The coronaviral genome encodes four major structural proteins: the spike (S) protein, the nucleocapsid (N) protein, the membrane (M) protein, and the envelope (E) protein, all of which are required to produce a structurally complete viral particle.

In general, pandemic viruses such as SARS-COV-2 have highlighted that certain microbial/viral characteristics make it extremely difficult to fully prevent microbial transmission via fomites (inanimate objects which carry infection) and/or aerosols in a high burden environment such as a hospital ward or nursing home. Such highly pathogenic characteristics include high microbial and/or viral load in the upper respiratory tract, the ability of infected persons to transmit the microbe/virus while asymptomatic, the ability of the microbe/virus to travel several meters in the air even if the subject merely exhales or speaks, and the ability of the microbe/virus to remain viable and infectious after hours in the air, and up to days on various surfaces. It seems that this makes this virus so virulent as it can live without a host for extended periods of time. The revealing fact is the difference of life sustainment on different surfaces and discovering what accounts for the variations of time the virus can live on different surfaces.

With such pathogens, it is inevitable that persons subject to high burden environments for an extended period will be subject io to and colonized by the pathogen. For those at risk, health authorities recommend the use of PPE (personal protective equipment) to reduce the risk of pathogenic transmission in aerosolized droplets. PPE requirements typically include either one or a combination of gloves, gowns, face shields, and masks (N95, surgical or community masks). PPE requirements in high burden environments can be extensive as suggested by a recent study at the Hospital Clinico San Carlos in Milan, which suggests that a 24 hour shift in a 12-bed intensive care unit (ICU) is staffed by 12 doctors, 32 nurses, 2 radiology technicians, 2 cleaning personnel and 2 consultants. In a 24 hour shift, these 50 providers would require 100 sets of gloves (to double glove), 50 gowns, 50 face shields and 50 masks. Extrapolating this Italian model to the whole United States, approximately 830′000 sets of gloves, 415′000 gowns, 415′000 face shields, and 415′000 masks would be required daily.

Clearly, this would put a great burden on the PPE supply chain, and health authorities are advocating for either a) reusing or b) manufacturing PPE from scratch, both raising concerns about the ability of PPE to be protective.

The antimicrobial and antiviral properties of various alcohols have been well known for years, and alcohol-based antiseptic liquid formulations designed to kill microbes on skin are commonly used in the hospital setting in addition to PPE to protect healthcare workers from microbial and viral exposure. Hands are a common vector for microbial spread, and as outlined by the WHO guidelines on hand hygiene in healthcare:

a) the hands offer an attractive environment for microbes/viruses to grow,

b) microbial contamination increases linearly with longer duration of patient care in the absence of proper hand cleansing,

c) the inanimate environment is commonly colonized by microbes/viruses (e.g., surfaces of phones, computer keyboards, and even PPE).

Not only can PPE act as a vector for infection, but during times of increased demand, such as the current global COVID-19 pandemic, the supply of unworn PPE can become quite limited.

In addition, the virus originates from a moist environment and become airborne. It carries an outer ultra-thin layer of water.

Therefore, the virus will survive on surfaces for several days.

It has been tested that when the virus lands on surfaces like PPE (N 95 and the like) it can stay infective for an average of 7 days and can be re-airborne.

The problem of the present invention is therefore to provide an antiviral and antibacterial composition made from safe ingredients to protect mouth and nose from viral infection.

The problem is solved by the composition according to the present invention. Further preferred embodiments are subject of the dependent claims.

It was found that the composition according to the present invention is an extremely powerful antiviral and/or antimicrobial agent, wherein the composition comprises at least
a positively charged natural or unnatural amino acid,
an organic acid
a cationic polymer, and
a zwitterionic surfactant.

It was found that the unique combination of the ingredients of the composition according to the present invention surprisingly create a synergistic effect to inactivate enveloped viruses and bacteria. These ingredients work selectively to inactivate the envelope virus by targeting the protein, the lipid and the amino acids of the viral membrane, spike and envelope. The strong cationic polymer of the composition interferes with the balance of the ionic charges of the virus thus attracting the virus and overwhelming the viral charges. This creates an imbalance in the equilibrium of viral ionic charges. Further, the organic acid and the amino acid interact with the protein of the infective viral RNA and disable it. Thus, the synergistic action of the ingredients of the composition according to the present invention allows to deactivate viruses and bacteria. The composition according to the present invention creates an active surface that can inactivate the virus and/or bacterium in minutes. The ingredients of the composition according to the present invention ensure in an aquatic status its dissociation and activities. Thus, the composition according to the present invention can, when applied to a carrier such as a mask, not only capture the virus but at the same time inactivate the virus permanently. In addition, once destroyed, they no longer adhere to the surface treated with the composition according to the present invention.

The composition according to the present invention can comprise up to 95% by weight of water. However, it can also be provided as a concentrate or even as essentially water-free composition.

Since all ingredients can be easily dissolved in water, the composition can be stored as concentrate and then be diluted before use if it is used for example as spray. However, for example, also saliva can be used to dissolve the active ingredients for example if the composition according to the present invention is provided as lozenges.

Since the ingredients according to the present invention are easily dissolved in water what is meant by the term “water-soluble” (thus, the indicated concentrations of all ingredients result in a clear solution at 25° C.) the moisture can connect with the water outer surface on the virus which is the key enabler of the capillary transmission of such ingredients into the surrounding water layer to effect the viral inactivation.

Preferably, the composition according to the present invention comprises an organic acid which is selected from the group consisting of malic acid, citric acid, lactic acid, acetic acid, glutamic acid, ascorbic acid and benzoic acid or a mixture thereof, preferably malic acid and/or glutamic acid.

Said acids are all GRAS compounds (generally regarded as safe compounds) and commonly used in preservatives, dyes, flavours and in food industry. Said organic acids lower the pH of the composition according to the present invention and act as an antiviral or antibacterial agent as a result of interacting with the proteins, the RNA and the lipids of the virus or the bacteria. These acid components can be added as powder to the composition according to the present invention. Especially malic acid and glutamic acid are preferred since they have a synergistic effect with the positively charged amino acid which is also contained in the composition according to the present invention. In addition, glutamic acid is known as humectant moisturizer and skin-conditioning agent which is an additional benefit.

Preferably, the composition according to the present invention comprises a positively charged amino acid which is selected from the group consisting of L-arginine, L-lysine, L-histidine, ornithine; 2,4-diaminobutanoic acid, 2,3-diaminopropanoic acid, 3-(aminoiminomethyl)amino-alanine, 2-amino-4-(aminoiminomethyl)aminobutanoic acid, N6-(aminoiminomethyly) lysine, 2-amino-7-(aminoiminomethyl)aminoheptanoic acid, 2,7-diaminoheptanoic acid, 2,8-diaminooxtanoic acid, 2,9-diaminononanoic acid, 2,10-diaminodecanoic acid, 4- (aminoiminomethyl)phenylalanine and 4-(aminoiminomethyl)aminophenylalanine, preferably the natural amino acids L-arginine, L-lysine and L-histidine. Best results could be obtained with L-arginine. L-arginine is the only amino acid with strong positive charge that remains protonated while binding to protein structure membranes. It binds to viral proteins, creates crowding and is able to suppress protein aggregation, thus making envelope viruses vulnerable to attack. In addition, due to its cationic charges it can interfere with the lipide membrane by causing pore formation in the lipid area, interfere and disrupt the flow of ions in the ion channel and with the phospholipid of the viral capsid.

The composition according to the present invention comprises a cationic polymer. The active cationic charges are provided by either a GRAS (Generally Recognized as Safe) material or industrial synthetic chemical compounds. Preferably the cationic polymer is selected from the group consisting of polyquaternium, fatty amines, polyethyleneimine or a copolymer thereof, cationic starch, metal cation components and mixture thereof. Most preferably, the composition according to the present invention comprises at least one polyquaternium.

Within the context of the present invention, the term polyquaternium (INCL designation) stands for polycationic polymers containing quaternary ammonium centres in the polymer which are typically used in the personal care industry. For example, PQ-1 through -47 of these polymers are listed in the Official Journal of the European Union, Commission Decision dated 9 Feb. 2006, 2006/257/EC. Even more polyquaternium polymers are known, and include in particular the following polyquaternium polymers:

| | | |
|---|---|---|
| PQ 2 | Mirapol ® A 15 | Rhodia (Solvay |
| | | Group) |
| PQ 4 | Celquat ® L 200 | Akzo Nobel |
| PQ 5 | Merquat ® 5 | Nalco Company |
| PQ 6 | Merquat ® 100 | Nalco Company |
| PQ 7 | Conditioneze ® 7 | Ashland Specialty |
| | | Ingredients |
| PQ 7 | Merquat ® 550 | Nalco Company |
| PQ 10 | Merquat ® 10 | Nalco Company |
| PQ 11 | Gafquat ® | Ashland Specialty |
| | | Ingredients |
| PQ 16 | Luviquat ® FC 370 | BASF Corporation |
| | Luviquat ® | |
| | Excellence | |
| PQ 17 | Mirapol ® AD 1 | Rhodia (Solvay |
| | | Group) |
| PQ 18 | Mirapol ® AZ 1 | Rhodia (Solvay |
| | | Group) |
| PQ 21 | Abil ® B 9905 | Evonik Industries |
| PQ 22 | Merquat ® 22 | Nalco Company |
| PQ 24 | Quatrisoft ® LM 200 | Dow Chemical |
| | | Company |
| PQ 28 | Gafquat ® HS 100 | Ashland Specialty |
| | | Ingredients |
| PQ 37 | Synthalen ® CR 3V | Sigma |
| PQ 39 | Merquat ® Plus 3330 | Nalco Company |
| PQ 44 | Luviquat ® | BASF Corporation |
| | UltraCare | |
| PQ 46 | Luviquat ® Hold | BASF Corporation |
| PQ 47 | Merquat ® 2001 | Nalco Company |
| PQ 53 | Merquat ® 2003 | Nalco Company |
| PA 55 | Styleze ® W Ashland | Specialty |
| | | Ingredients |
| PQ 68 | Luviquat ® Supreme | BASF Corporation |
| PQ 69 | AquaStyle ™ 300 | Ashland Specialty |
| | | Ingredients |
| PQ 86 | Luviquat ® Advanced | BASF Corporation |
| PQ 95 | Polyquart ® | Cognis Corporation |
| | Ecoclean | (BASF) |

Within the context of the present invention, the term metal cation components stands for colloidal systems comprising a metal ion which is stabilized by a tenside layer such as colloidal silver or colloidal copper. Most preferably, said metal cation components are present together with a further cationic polymer such as polyethyleneimine. For example, a combination of polyethyleneimine and colloidal silver results in a higher antibacterial and/or antiviral log than is these components are used alone.

The cationic polymer can also be cationic starch. Cationic starch made from starch granules reacted with quaternary ammonium yielding a continuous positive charge independent of pH. Many different commercially available cationic starches can be used for the present invention. The example includes CHARGEMASTER cationic starch lines (CHARGEMASTER line of cationic starches available from Grain Processing Corporation of Muscatine, Iowa). Chargemaster L340 is especially preferred. Cationic starch is non-toxic and can be produced in food grade, which is of course a big advantage, in particular if the composition according to the present invention is orally applied.

In one embodiment of the invention, the cationic polymer is a linear or branched polyethyleneimine which can have a high molecular weight (25 kDa) or a low molecular weight (1.8 kDa). Preferably, it is a branched polyethyleneimine comprising repeating units composed of ethylene diamine groups. It can contain primary, secondary and tertiary amino groups. Said branched polyethyleneimide can be at least partly crosslinked, preferably with a crosslinker selected from the group consisting of phthalaldehyde and PEG since these combinations significantly increase or even double the efficacy of the composition according to the present invention on surfaces.

In some embodiments, the hydrophobic polycationic polymer is an N-alkylated polyethylenimine with various alkyl chain lengths, such as N,N-dodecyl,methyl-polyethylenimine or N,N-hexyl,methyl-polyethylenimine. In other embodiments, the polymer is a poly(4-vinyl-N-alkylpyridine).

In addition, the composition according to the present invention comprises a zwitterionic surfactant. A zwitterionic surfactant has a positive and a negative charge and thus is less sensitive to pH changes. The zwitterionic surfactant interacts with both the hydrophobic and the hydrophilic sides of the amino acids which compose the proteins of the viral membrane and of the envelope protein, thus resulting in the viral protein disintegration. A preferred zwitterionic surfactant is cocamidopropyl betaine. Due to its long hydrocarbon chain, it can interact with the lipids and its polar head can interact with the viral ionic charges.

It could be shown that the effect of zwitterionic surfactants can be enhanced by the addition of non-ionic surfactants such as polysorbates resulting in a better membrane lipid and protein solubility.

The presence of the positively charged amino acid and of the organic acid has the effect that they buffer the composition according to the present invention. It has been found that the composition of the present invention has preferably a pH above 5.5, in particular for the inactivation of SARS-COV-2, since the proteins of the envelope and the spike have an isoelectric point of 5.5.

In one embodiment, the composition according to the present invention comprises L-arginine as positively charged amino acid and malic acid and/or glutamic acid as organic acid, preferably in a ratio of 1:10 to 10:1. The combination of L-arginine with one or both of said organic acids significantly increase the solubility of proteins by about factor 6 due to increased hydrogen bonding thus increasing the interaction between said ingredients and the surface of the viral protein.

The composition according to the present invention can include a humectant to provide skin moisturizing, skin softening, skin barrier maintenance, anti-irritation, or other skin health benefits. Some non-limiting examples of humectants include hydroxyethyl glycerine, urea, agarose, urea, 5-Oxo-L-prolin, fructose, glucose, honey, lactose, maltose, polyethylene glycol, sorbitol and mixtures thereof.

Preferably, the composition according to the present invention is free of ethanol. Ethanol attacks and destroys the envelope protein that surrounds some viruses, including coronaviruses.

In contrast, hand sanitiser needs to contain at least 60% alcohol in order to kill most viruses. However, such high levels of ethanol dry the skin and in a worse case cause dermatitis, especially in low humidity climates or during the “dry” months of the year.

In addition, the composition according to the present invention can additionally comprise a cationic surfactant to increase the positive charge. Examples of cationic surfactants are cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethyibenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimefhylammonium chloride, and the corresponding hydroxides thereof.

Especially good results could be obtained with a composition comprising at least a total of 0.02% to 8% by weight of a positively charged natural or unnatural amino acid, 0.02 to 8% by weight of an organic acid, 0.02 to 5% by weight of a cationic polymer, and 0.02% to 8% by weight of a zwitterionic surfactant. Preferably such a composition comprises up to 92% by weight of water. Especially preferred is a composition comprising at least 0.02% to 8% by weight of L-arginine, 0.02 to 5% by weight of malic acid or glutamic acid or a mixture thereof, 0.02 to 5% by weight of polyquaternium and 0.02 to 8% by weight of a cocamidopropyl betaine. Preferably, such a composition comprises up to 92% by weight of water.

The composition according to the present invention can inactivate a broad variety of bacteria and viruses, in particular viruses selected from the group consisting of corona virus, influenza virus, human rhinovirus (HRN), parainfluenza virus (PIN), respiratory syncytial virus (RSN), adenovirus, metapneumovirus, and rhinovirus, and especially SARS-COV-2 or a mutant thereof. The composition of the present invention is especially active against airborne viruses.

The composition according to the present invention can be applied in different formulations such as a spray, a pre-spray gel (spray and dry system), a water soluble pod, a mist, a strip or a lozenge.

Water soluble pods allow to store the composition according to the present invention as concentrate while the bottle can be reused thereby avoiding shipping and paying for water and saving on the cost of individual plastic bottle and spray systems. In addition, it has a significant sustainability value. Water soluble pods containing the composition of the present invention can be applied alone or with other ingredients such as laundry detergents or dish washing detergents. The composition of the current invention within the pods are capable of activating fabrics such as hospital bed linins in bulk thereby protecting patents against bacteria and virus. In addition, the composition of the present invention can be used to activate dishes and utensils as well as tools or any object placed within the washing equipment. The composition of the present invention can be applied before during or after the rinse cycle of the washing process or during drying. The composition of the present invention can have fragrances added to enhance comfort and smell of the fabrics.

Wash systems such as sinus wash systems are known to the skilled person. The composition according to the present invention can directly be used as a wash system or it can be added as a concentrate to the usual saline solution.

In one embodiment of the present invention relates a carrier which is coated with a composition according to the present invention or its essentially water-free dried form. The term essentially water-free means a water content of less than 10%.

Spray-drying is a method in which a composition is sprayed by a device for preparing fine particles on the surface of a carrier and subsequent drying by evaporation of moisture. Thus, a carrier which is coated with the composition according to the present invention in its essentially water-free form means that the carrier is covered with a thin layer comprising the ingredients of the composition according to the present invention, wherein the water has been partly or fully evaporated. Interestingly, the composition according to the present invention is active in its dried form. Since the corona virus is an airborne virus, its water layer on the virus particle will reactivate the composition according to the present invention and capture and inactivate the virus.

Carriers coated with the composition according to the present invention have preferably more than 15 millions, preferably more than 50 millions and most preferably more than 100 millions positively charged ions per square centimetre and adhere the negatively charged viruses and bacteria. On contact with this surface, viral protein capsids or envelopes are disrupted.

The term carrier within the context of the present invention stands for any surface that could come into contact with the virus or the bacteria. Preferably, said carrier comprises or consists of molded fiber, plastics, non-wovens, foam and open cell foam, rubbers and textiles. Due to the composition according to the present invention, it is possible to create a smart active surface mask that can allow breathing, block penetration and inactivate viruses. Especially, it is no longer necessary to use N95 masks, which can cause difficulties in breathings and are universal with one size fits all creating significant discomfort, skin irritation and inconvenience during use. Actually, new recent regulations are limiting the continuous use of N95 directly on the face for not more than 75 minutes before removal for a short period to allow breathing fresh air.

Carriers, such as masks made of molded fibers are especially preferred. They are made from recyclable pulp of paper which are locally available. Such carriers can be ergonomically optimized with specific size and shape, for example addressing the difference in physiognomy of male and female.

Furthermore,Laminating an inner clear plastic film on the inside of the mask will allow for friendly interaction with skin and face while providing excellent protection. In addition, Such clear film can be integrated and positioned inside the mask to offer a see through of the mouth while offering protection and can be protected from viral accumulation by using an antiviral spray treatment. The nose area can be equipped by appriately treated menbranes to allow continuous fresh air breathing. The capital costs for molds are very low allowing a production of said carriers also in poor countries. The surface structure of the molded fibers allow a good retention of composition according to the present invention.

The coated surface while dry is still receptive to absorb water, humidity, moisture from the air as well as from breathing through the nose or mouth. The PPE, masks and nasal devices can be pre-treated, and its surface will be immediately active.

The composition according to the present invention can also be used as a surface treatment. The surface treatment will block, capture, and kill enveloped viruses and bacteria providing maximum protection against transfer and infection in wet or dry environments. Such surfaces include doorknobs, elevator buttons, staircase railings, telephone sets, computer keyboards and water taps which all commonly serve as vectors for viral transmission.

The carrier which can be treated or pre-treated with the composition according to the present invention is preferably a personal protection equipment, and most preferably selected from the group consisting of air filters, personal protective equipment, N95 surgical mask, community mask, textile mask, foam mask, Bandana mask, molded fiber mask, foam textiles, cotton textiles, cellulose textiles, composites, nasal inserts, foam nasal inserts, nasal filters, nasal screens or nasal filters, air filters, surgical gowns, coverings and wipes.

All ingredients of the composition according to the present invention are water-soluble or water-dissolvable. Therefore, the carrier can be washed in the washing machine or by hand, thereby removing the composition according to the present invention. Afterwards, the carrier can be dried, for example air-dried in a clean space before treating it again with the composition according to the present invention. Thus, the composition according to the present invention allows to use reusable personal protective equipment.

The pre-treated carrier with plasma/Corona resulting in active surface ion can be stored in a modified atmospheric packaging, gas barrier plastic, foils and nitrogen rich environments or vacuum type packaging in order to protect the treated surface before use.

If the carrier is a personal protection equipment comprising a filter system, said filter system can comprise activated carbon and an acidic protein fibril membrane. The composition of the present invention enhances the cationic nature of the fibril membrane, and thus increases the efficacy of the filter system. In addition, such a fibril protein-based membrane may additionally be coated or impregnated with polyethylenimine (PEI)/branched polyethylenimine (BPEI), or mixed with colloidal silver and/or colloidal copper ions to further increase the positive charge on the surface of the carrier, which acts like a positive magnet for the negatively charged viruses and bacteria.

The composition according to the present invention can also be applied topically or orally. Due to its safe ingredients, it can be sprayed on the naked skin around the mask as well as on the inside of the mask including the nose, throat mouth and the whole respiratory system. The composition can be applied to the human respiratory system, from nose and mouth to the lungs, via standard delivery techniques of consumer and medical products, including sprays, food-based preparations, lozenges, oral strips, solutions for nasal irrigation systems (nasal wash) and fine mist. Especially preferred, the composition according to the present invention is used in a system for nasal irrigation since it is medically proven that infection through nasal inhalation is 10′000 times more often than through the mouth. A nasal rinse with the composition according to the present invention can significantly reduce the virus load in this area.

In order to protect the nasal system, an active anti-viral device capable of capturing the virus particles floating in the inhaled air can be used. Such nasal systems are known in the art, for example as cosmetic nose protector, nasal air filters (U.S. Pat. Nos. 6,962,156; 6,971,387; 6,981,501), nasal tampon or as nose dilators for snoring (for example comprising an existing flexible frame and an exchangeable filter). Said systems can be partly or fully treated with the composition according to the present invention, and therefore inactivates the virus when inhaled through the nasal system. The manufacturing process for such a system can for example involve the production of the foam by a chemical reaction process and then removing the cell walls within the foam by a thermal or chemical process thereby producing reticulated foam. The reticulated foam consists of a three-dimensional matrix with voids and intricacies within the skeletal structure. Preferably, the foam is an open cell reticulated polyurethane foam of low density and light weight. Reticulating the foam allows for managed cell numbers, its design, shape, and location within the foam structure. Alternatively, a polyether or polyester foam may be used. The porosity of such a foam can range from 10-100 pores per inch. Alternatively, the system can be made of molded fibers. Dependent on the selected porosity or the nature of the molded fiber, the structure allows for high breathability and high retention of the composition according to the present invention. Such nasal systems are extremely cost effective, safe, highly effective, and extremely sustainable. They can be used for example in all indoor activities such as in restaurants, theatres, schools as well as in public transport and airplanes.

In addition, such a nasal system can be pre-treated with cold atmospheric plasma. Cold atmospheric plasma and/or corona is known to the skilled person and allows to increase the cationic charge and the electrostatic charges of the surface A metallic stearate, preferably selected from the group consisting of magnesium stearate, calcium stearate and zinc stearate can be added during the foaming process or sprayed pre plasma treatment to help maintain and extend the ionic charges created. In addition, such substrates can be treated with a food grade silicone base material preferably polydimethyl siloxane PMDS to enhance the ionic density and retention. These treated foam structures have low odour and in some embodiments are made to fit certain medical specifications.

The composition according to the present invention may contain other additives typically used in cosmetic medical applications like gelling agents, film forming agents, coalescing agents such as polyvinyl acetate(PVA), methocel, carboxymethyl cellulose, preservatives such as benzalkonium chlorides, suspending agents, thickening agents, emollients, and other ingredients without impacting the antiviral potency of the key active ingredients. Such additional ingredients are known to the skilled person.

In a further embodiment of the present invention, the carrier is additionally treated with cold atmospheric plasma. Cold atmospheric plasma is known to the skilled person and allows to increase the cationic charge of the surface. The presence of a metallic stearate such as magnesium stearate, calcium stearate or zinc stearate on the carrier or the composition according to the present invention can stabilize electrostatic charges and result in intensified and retained anti-viral properties.

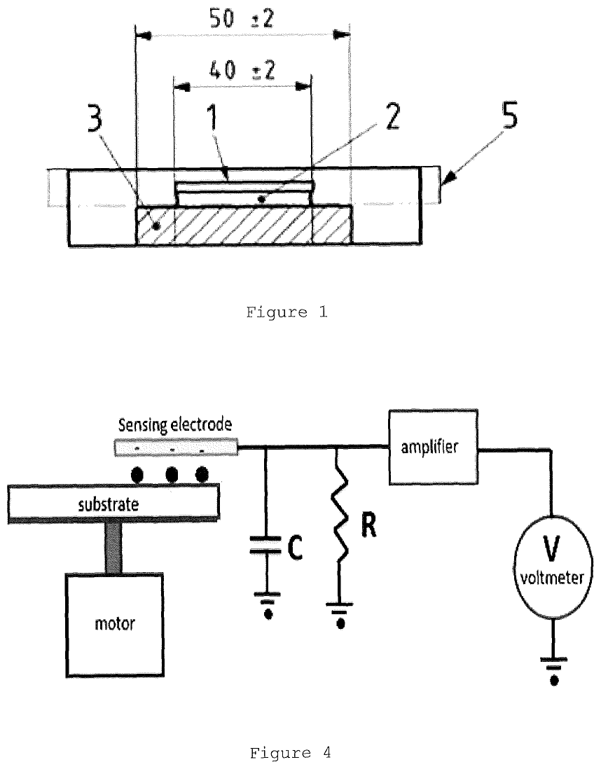

FIG. 1 shows a schematic diagram of the experimental setup;

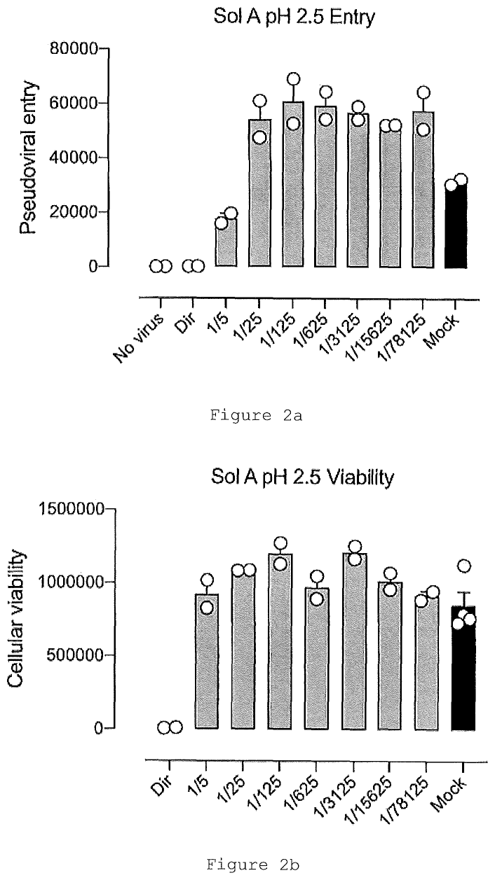

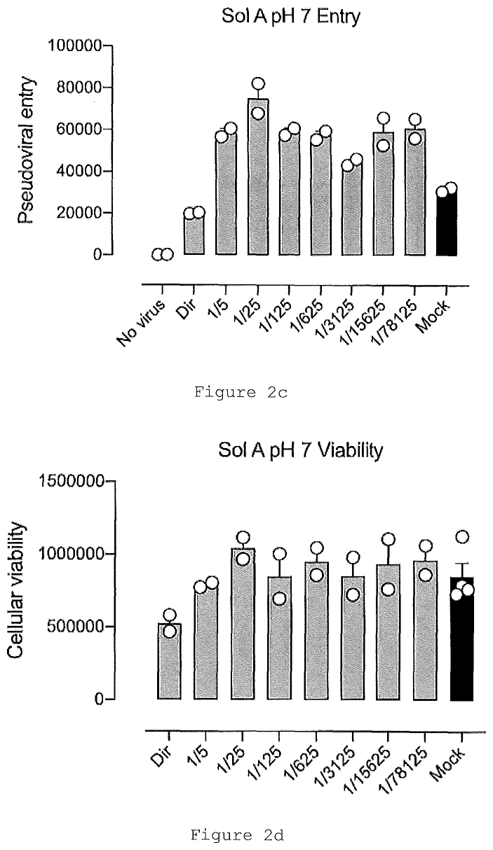

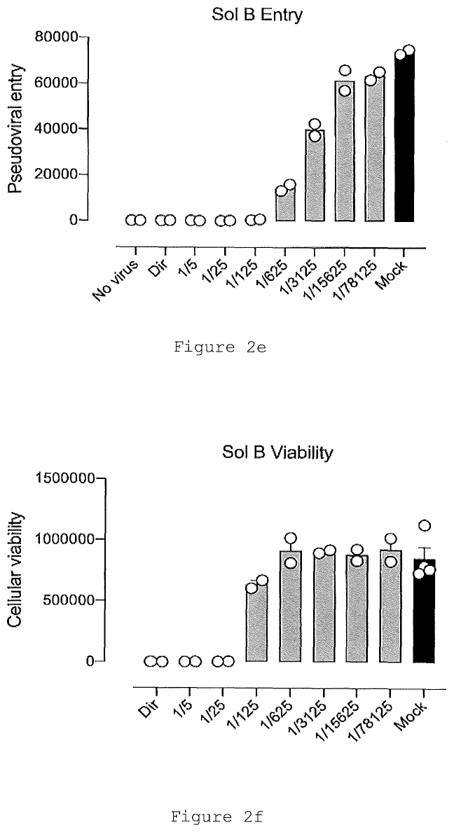

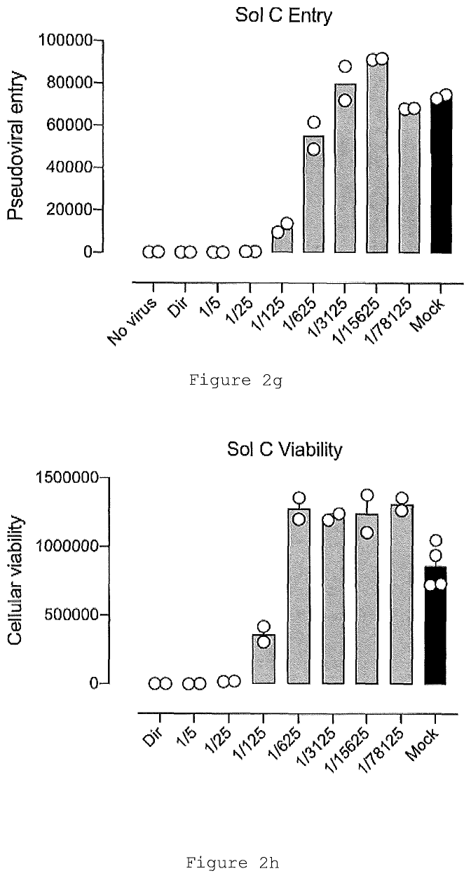

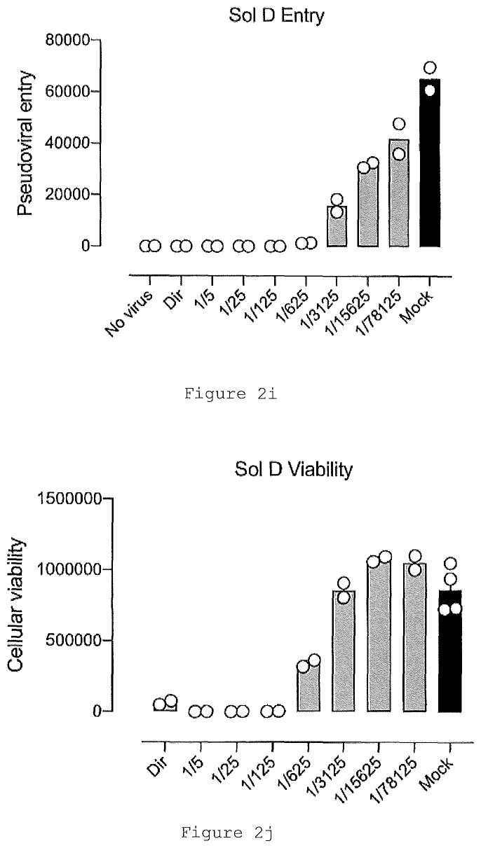

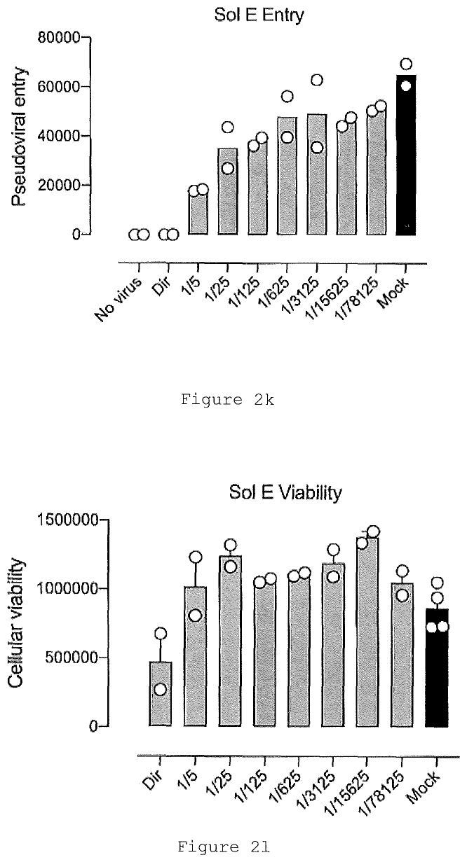

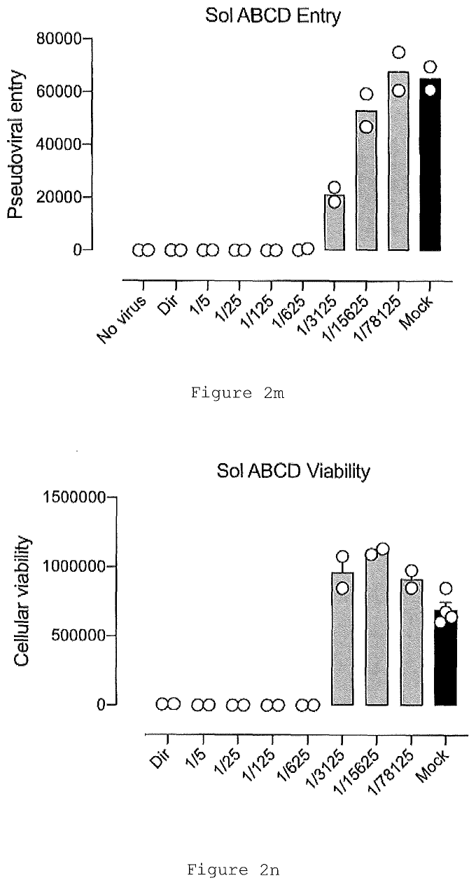

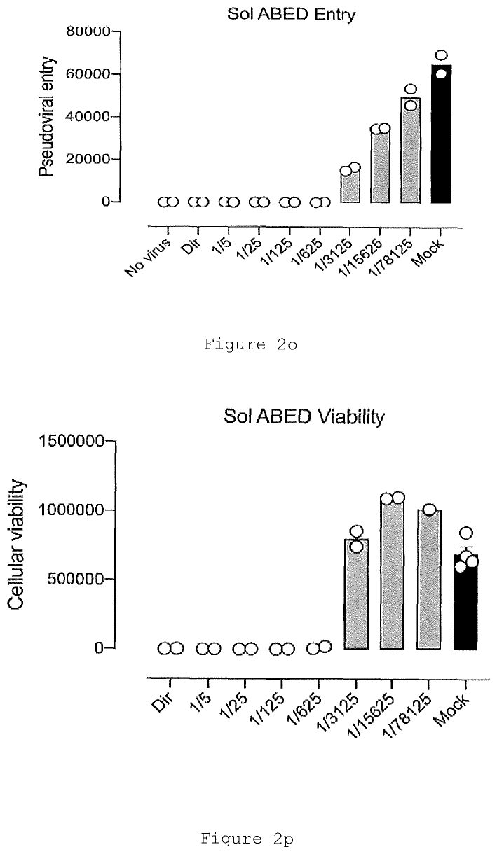

FIG. 2 shows the antiviral activity of solutions and mixes inhibiting SARS-CoV-2 entry.

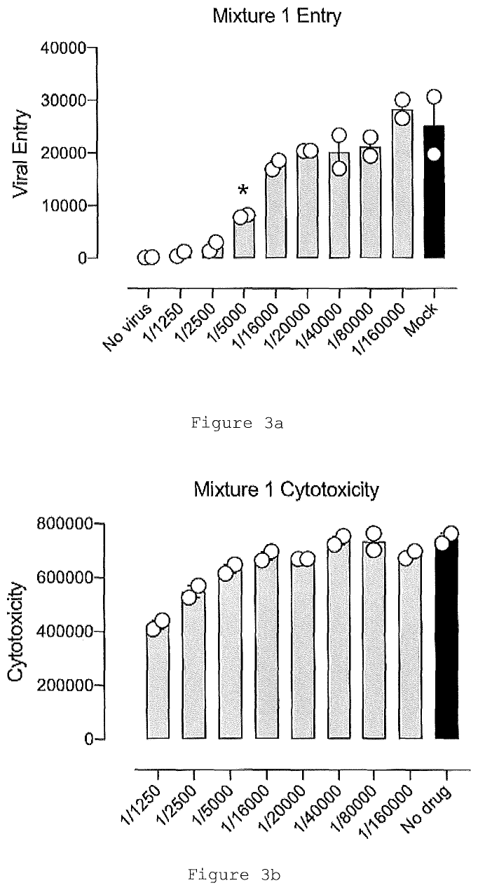

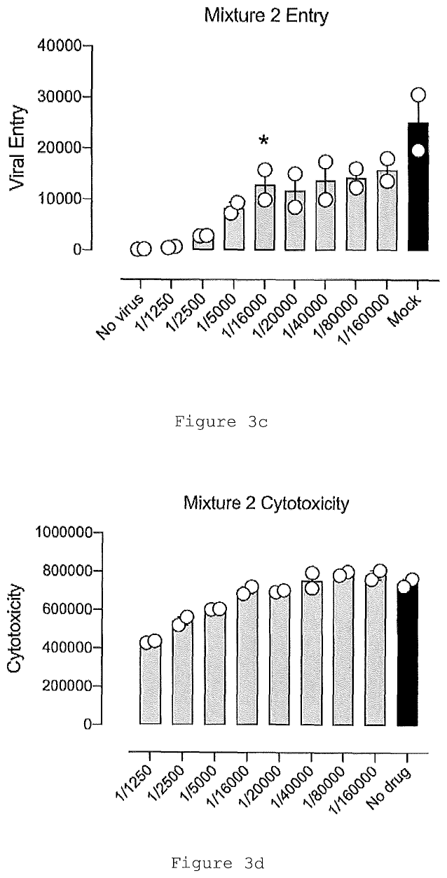

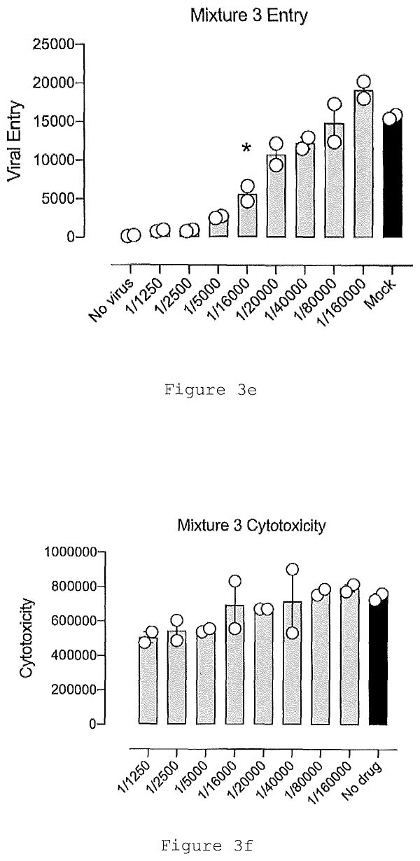

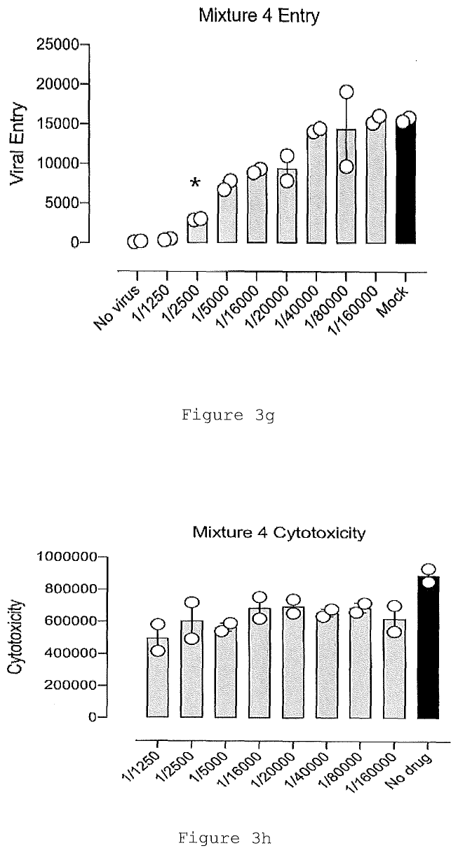

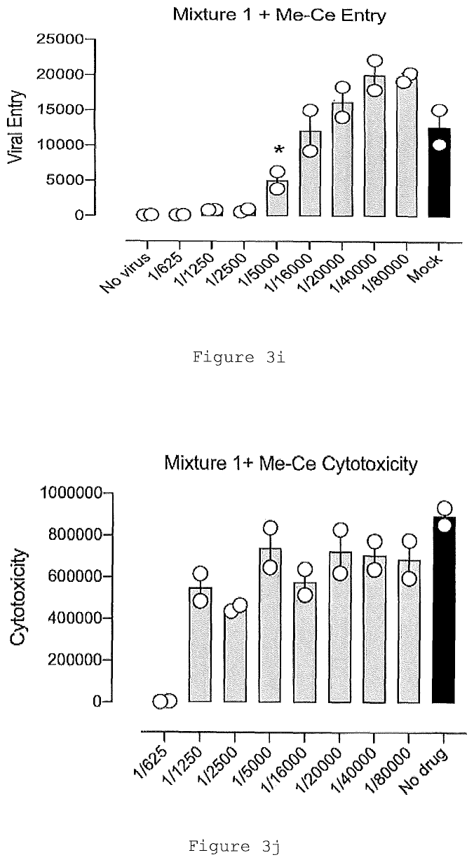

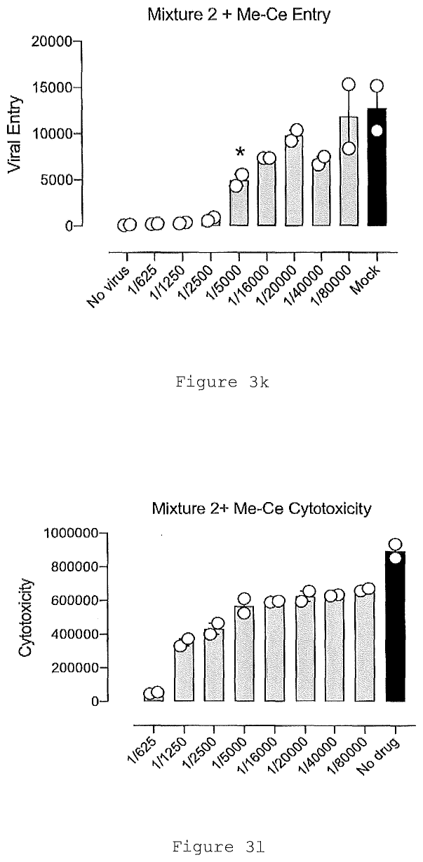

FIG. 3 shows the antiviral activity of mixes inhibiting SARS-CoV-2 entry.

FIG. 4 shows a schematic diagram of the experimental setup.

### EXAMPLES

### Example 1: Iso like Experiment on Coated Petri Dishes

Step 1 Prepare 400 ul of R18 rhodamine fabled inactivated virus inoculum solution in PBS for each sample.

Step 2 Apply the inoculum on the sample and sandwich it with LOPE inert film as shown in FIG. 1 (ISO 21702):
A Petri dish 5 is coated with the composition according to the present invention and dried to form an antiviral surface 3.
Artificial inactivated enveloped coronavirus sample 2 having the same composition of covid 19 virus but the mRNA genome was removed and replaced with a fluorescent dye is added to the antiviral surface 3.
Plastic film 1 to cover the petri dish antiviral surface once the viral solution is applied.

Step 3 Incubate the samples for 24 hours at room temperature and in dark environment.

Step 4 Add 10 ml of PBS to each sample to recover the inoculum.

Step 5 Pipette 2 ml of the recover mixture in a transparent cuvette (2 replicates per sample).

Step 6 Measure emission spectra with fluorometer (excitation wavelength 560 nm, emission measure from 580 nm to 650 nm).

Antiviral efficacy of the solution was determined by using the above test procedure.

The interaction and the disintegration of the virus is determined by measuring the fluorescent concentration on the antiviral surface resulting from viral disintegration. the range is defined by the max amount of fluorescent dye represented as (PC) and no dye as (NC) indicating no interaction. The graph below shows that the formulations demonstrated efficacy against the virus.

| | | Florescent |
|---|---|---|
| | | intensity in |
| Formulation | PH | MM |
| Malic acid 5% | 2.5 | 7.5 |
| Luviquat 5% | | |
| Cocamidopropyl 5% | | |
| Malic acid 5% | 5.5 | 7.4 |
| Luviquat 5% | | |
| Cocamidopropyl 5% | | |
| L-Arganine 10% | | |
| Malic acid 5% | 2.5 | 7.7 |
| Cationic starch5% | | |
| Cocamidopropyl 5% | | |
| L-Arganine 0% | | |
| PVA 10% | | |
| Malic acid 5% | 5.5 | 7 . 6 |
| Luviquat 5% | | |
| Cocamidopropyl 5% | | |
| PVA 10% | | |
| L-Arganine 10% | | |
| Malic acid 5% | 2.5 | 7.4 |
| Luviquat 5% | | |
| Cocamidopropyl 5% | | |
| PVA 10% | | |
| Malic acid 5% | 5.5 | 7.5 |
| Luviquat 5% | | |
| Cocamidopropyl 5% | | |
| Glycerin 2% | | |
| PVA 10% | | |
| Control (no effect on | | 0.9 |
| disintegration) | | |
| Control (maximum effect | | 7.7 |
| on disintegration ) | | |

### Example 2: Inhibition of SARS-CoV-2

Cell Cultures: Vero E6 cells (ATCC CRL-1586) were cultured in Dulbecco's modified Eagle medium, (DMEM) with 10% fetal bovine serum, 100 IU/ml penicillin and 100 μg/ml streptomycin (all from Invitrogen). HEK-293T overexpressing the human ACE2 were kindly provided by Integral Molecular Company and maintained in DMEM (Invitrogen) with 10% fetal bovine serum, 100 IU/ml penicillin and 100 μg/ml streptomycin, and 1 μg/ml of puromycin (all from Invitrogen).

Pseudovirus production: HIV-1 luciferase reporter pseudoviruses expressing SARS-CoV-2 Spike protein were generated using two plasmids. pNL4-3.Luc.R-.E- was obtained from the NIH AIDS repository. SARS-CoV-2.SctA19 was generated (Geneart) from the full protein sequence of SARS-CoV-2 spike with a deletion of the last 19 amino acids in C-terminal, human-codon optimized and inserted into pcDNA3.4-TOPO 1. Spike plasmid was transfected with X-tremeGENE HP Transfection Reagent (Merck) into HEK-293T cells, and 24 hours later, cells were transfected with pNL4-3.Luc.R-.E-. Supernatants were harvested 48 hours later, filtered with 0.45 pm (Millex Millipore) and stored at −80° C. until use. Viruses were titrated in HEK-293T overexpressing human ACE2 to use an equal amount of fusogenic viruses.

Pseudovirus assay. HEK-293T overexpressing the human ACE2 were used to test provided mixes and their vehicles at the indicated dilutions. A constant pseudoviral titer was used to pulse cells in the presence of the samples. After 48h post-inoculation, cells were lysed with the Bright Glo Luciferase Assay system (Promega). Luminescence was measured with an EnSight Multimode Plate Reader (Perkin Elmer). To detect any associated cytotoxic effect, mix formulations were also tested with media, and were equally cultured on cells but in the absence of pseudovirus. Cytotoxic effects of these products were measured 48h post-inoculation, using the CellTiter-Glo luminescent cell viability assay (Promega).

Sample Preparation:

| Mixture | 1 | 1.1 | 2 | 2.1 | 3 | 4 |
|---|---|---|---|---|---|---|
| Malic acid | 1% | 1% | 1.0% | 1.0% | 1.0% | 1.0% |
| Cocamido-propyl | 2.5% | 2.5% | 2.5% | 2.5% | 2.5% | |
| Lecithin | | | | | | 2.5% |
| Luviquat | 2.4% | 2.4% | 2.5% | 2.5% | 1.0% | |
| b-cyclodextrin | | | | | | 2.5% |
| Benzalkonium | 2.5% | 2.5% | 0.75% | 0.75% | 0.75% | 0.75% |
| chloride | | | | | | |
| L-Arginine | 2.0% | 2.0% | 2.0% | 2.0% | 2.0% | 2.0% |
| Me-cellulose | | 0.2% | | 0.2 | | |

Results

We have tested the capacity of the provided mixes to inhibit

SARS-CoV-2 entry into target cells. We employed a luciferase-based assay, using a reporter lentivirus pseudotyped with the spike protein of SARS-CoV-2, which allows the detection of viral fusion with target HEK-293T cells expressing human ACE2 receptor. A constant concentration of the reporter pseudovirus containing the SARS-CoV-2 original Spike protein was mixed with increasing concentrations of the indicated CPC-containing mouth rinses, or their corresponding vehicles, and added to the target cells. To control for any induced cytotoxicity of the mixes, target cells were also cultured with increasing concentrations of the indicated products in the absence of pseudoviruses. All solutions but A at pH 7, C and E were able to inhibit viral fusion in a dose dependent manner at concentrations where no cytotoxic effects were observed (FIG. 2). The combinations of these solutions (mixtures) were very efficacious at inhibiting viral entry at non cytotoxic concentrations. New mixes including active solutions where prepared, and were able to inhibit viral fusion in a dose dependent manner at concentrations where no cytotoxic effects were observed (FIG. 3). These results indicate that solutions and their mixes able to block SARS-CoV-2 viral entry into target cells.

Figures

FIG. 2. Antiviral activity of solutions and mixes inhibiting SARS-CoV-2 entry. Viral entry inhibition on target HEK-293T cells expressing ACE2 exposed to a fixed concentration of SARS-CoV-2 in the presence of increasing concentrations of solutions and their mixes. Cytotoxic effect on HEK-293T cells expressing ACE2 cells exposed to increasing concentrations of solutions and mixes in the absence of pseudovirus is also shown (right panels).

FIG. 3. Antiviral activity of mixes inhibiting SARS-CoV-2 entry. Viral entry inhibition on target HEK-293T cells expressing ACE2 exposed to a fixed concentration of SARS-CoV-2 in the presence of increasing concentrations of mixes. Cytotoxic effect on HEK-293T cells expressing ACE2 cells exposed to increasing concentrations of mixes in the absence of pseudovirus is also shown (right panels).

### Example 3: Measurement of the Active Surface Ionic Charge Density

The amount of surface charge density can be determined by means of measurement of induced image charges in a sensing electrode. The treated surface repeatedly moves close to and away from a sensing electrode and the induced image charge creates an AC electrical current in the circuitry connected to the sensing electrode. The induced current is measured and is proportional to the surface charge.

The apparatus consists of a sample spinner, contained in a metal box, sensing electrode and Keithley 823 nanovolt amplifier (FIG. 4)

C and R are capacitance and resistance of the input circuitry of the amplifier. Input capacitance was measured 80pF and input resistance is 50 MOhm.

The sensing electrode is made of 1.3 mm diameter copper wire. When the metal box top is in closed position, the sensing electrode is about 1.5mm above the sample surface. One half of the sample substrate is treated, and another half is untreated. During the sample spinning treated and untreated surface repeatedly move under the sensing electrode. The surface charge is calculated using the following formula:

Q=V*C/A

Where Q is charge per unit area, V is measured voltage on the sensing electrode and A is the area of the sample under the sensing electrode.

Sample Preparation:

Paper or corrugated substrate disks are prepared in approximately 2.5″ circular in diameter. Treated samples are attached to 50% of the diameter by use of adhesive or tape. The apparatus detects Ionic charges by sensing the differential of charges on a treated and untreated surface. A disk is prepared wherein half of the disk is treated and the other is not. As the disk is rotated the sensing electrode detects the charge differential.

Sample Preparation:

Paper or corrugated substrate disks are prepared in approximately 2.5″ circular in diameter. Treated samples are attached to 50% of the diameter by use of adhesive or tape.

The apparatus detects Ionic charges by sensing the differential of charges on a treated and untreated surface. A disk is prepared wherein half of the disk is treated and the other is not. As the disk is rotated the sensing electrode detects the charge differential. Utilizing the apparatus, and following the identical testing procedure, we tested numerous samples of the “Livinguard” commercial mask. The average Ionic density on the surface, is indicated in the table below.

### EXAMPLES

| | | Charges |
|---|---|---|
| | Charge | density per |
| | per unit | cm² in |
| Composition | area | millions |
| Silicone (industrial) | 2.94E+08 | 294 |
| Malic acid | 9.80E+07 | 98 |
| Luviquat | | |
| Cocamidopropyl | | |
| Malic acid | 1.58E+07 | 15.8 |
| Luviquat Cocamidopropyl | | |
| L-Arganine | | |
| Malic acid | 1.35E+08 | 135 |
| Cationic starch | | |
| Cocamidopropyl | | |
| L-Arganine | | |
| Malic acid | 1.67E+08 | 167 |
| Luviquat, | | |
| Cocamidopropyl | | |
| Malic acid | 4.27E+08 | 427 |
| Luviquat | | |
| Cocamidopropyl | | |
| PVA | | |
| Malic acid | 2.62E+08 | 2 62 |
| Luviquat | | |
| Cocamidopropyl Glycerin | | |
| Livinguard (Commercial | | 20 |
| Mask) | | |

### Example 4

Two bacterial strains were tested against three different compounds provided. The Gram-negative bacterial strain E. coli K12 was grown in LB media, and the Gram-positive strain Staphylococcus aureus 113 was cultured in BHI media overnight prior to antimicrobial test. Bacterial density was determined by OD600 measurements and adjusted to approximately 108 bacterial cells per mL with broth media, respectively. Equal volume of compound solutions and bacterial cells were mixed and incubated at 37° C. To determine the killing efficacy, 20 μL of the mixed bacterial suspension was numerated at 1 h, 3 h, 6 h and 30 h after incubation with corresponding compounds, by distributing on an LB agar plate (for E. coli) and BHI agar plate (for S. aureus) at 10-fold serial dilutions. The plates were further incubated at 37° C. for 24 h and bacteria viability was determined by counting the colony forming units (CFU).

Testing the formulation samples for antibacterial efficacy shows that the inhibition is effective but in a slow kinetic manner. Bacterial count reduction after a 2 hour incubation was low. However, almost no survived bacteria can be detected after a 24 hour incubation. Reduction time can be improved by increasing the concentration of the components.

1. Use of a composition as antiviral and/or antimicrobial agent, wherein the water soluble composition comprises at least
a positively charged natural or unnatural amino acid,
an organic acid,
a cationic polymer, and
a zwitterionic surfactant.

2. Use according to claim 1, wherein the composition comprises up to 92% by weight of water

3. Use according to claim 1, wherein the organic acid is selected from the group consisting of malic acid, citric acid, lactic acid, acetic acid, glutamic acid, ascorbic acid and benzoic acid or a mixture thereof.

4. Use according to claim 1, wherein the positively charged amino acid is selected from the group consisting of L-arginine, L-lysine, L-histidine, ornithine, 2,4-diaminobutanoic acid, 2,3- diaminopropanoic acid, 3-(aminoiminomethyl)amino-alanine, 2-amino-4-(aminoiminomethyl)aminobutanoic acid, N6-(aminoiminomethyly) lysine, 2-amino-7- (aminoiminomethyl)aminoheptanoic acid, 2,7-diaminoheptanoic acid, 2, 8-diaminooxtanoic acid, 2, 9-diaminononanoic acid, 2,10-diaminodecanoic acid, 4- (aminoiminomethyl)phenylalanine and 4-(aminoiminomethyl)aminophenylalanine.

5. Use according to claim 1, wherein the cationic polymer is selected from the group consisting of polyquaternium, fatty amines, polyethyleneimine or a copolymer thereof, cationic starch, metal cation components and mixture thereof.

6. Use according to claim 5, wherein the cationic polymer is a branched polyethyleneimine which is at least partly crosslinked.

7. Use according to claim 1, wherein the composition additionally comprises a humectant and or antistatic material.

8. Use according to claim 1, wherein the composition comprises L-arginine as positively charged amino acid and malic acid and/or glutamic as organic acid.

9. Use according to claim 1, wherein the composition comprises at least
0.02 to 8% by weight of a positively charged natural or unnatural amino acid,
0.02 to 8% by weight of an organic acid,
0.02 to 5% by weight of a cationic polymer, and
0.02 to 8% by weight of a zwitterionic surfactant.

10. Use according to claim 1, wherein the composition is free of ethanol.

11. Use according to claim 1 to inactivate a virus and a bacteria selected from the group consisting of corona virus, influenza virus, human rhinovirus (HRN), parainfluenza virus (PIN), respiratory syncytial virus (RSN), adenovirus, metapneumovirus, rhinovirus, and SARS-COV-2 or a mutant thereof.

12. Use according to claim 1, wherein the composition is provided as a spray, a pre-spray gel, a water soluble pod, a mist, a strip, a lozenge and wash system.

13. A carrier which is coated with a composition according to claim 1 or its essentially water-free dried form.

14. The carrier according to claim 13, wherein the carrier is selected from the group consisting of air filters, personal protective equipment, N95 surgical mask, community mask, textile mask, foam mask, Bandana mask, molded fiber devices, foam textiles, cotton textiles, cellulose textiles, composites, nasal inserts, foam nasal inserts, nasal filters, nasal screens, nasal filters, air filters, surgical gowns, coverings and wipes.

15. The carrier according to claim 3 wherein the carrier is additionally treated with cold atmospheric plasma and or corona to create surface electrostatic and cationic charges.
